(19) United States
(12) Patent Application Publication
(10) Pub. No.: US 2008/0015549 A1
(43) Pub. Date: Jan. 17, 2008

(54) System For Processing Medication Restriction Information

(75) Inventor: Rex Wendell Maughan, Murray, UT (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., MALVERN, PA (US)

(21) Appl. No.: 11/778,292

(22) Filed: Jul. 16, 2007

Related U.S. Application Data
(60) Provisional application No. 60/807,515, filed on Jul. 17, 2006.

(51) Int. Cl. A61K 9/22

(52) U.S. Cl. 604/890.1

(57) ABSTRACT

A system integrates drug cumulative dosage monitoring, clinical information storage and workflow providing clinical notifications and worklists. The system monitors patient cumulative medication intake and includes at least one repository of information identifying medications and associated cumulative dosage limits for different time periods and for different types of patient. A monitoring processor, in response to a detected administration event indicating an additional dosage of a particular medication is to be administered to a particular patient, automatically determines whether a cumulative dosage, including the additional dosage, of the particular medication of the particular patient exceeds a cumulative dosage limit over a time period using information derived from the at least one repository. A notification processor automatically initiates generation of an alert message to at least one healthcare worker using a selected communication method The selected healthcare worker and the selected communication method is identified using predetermined notification configuration information.

This is a non-provisional application of provisional application Ser. No. 60/807,515 filed Jul. 17, 2006, by R. W. Maughan.

### FIELD OF THE INVENTION

This invention concerns a system for automatically monitoring patient cumulative medication intake and associated alert message and notification processing.

### BACKGROUND OF THE INVENTION

Medications have restrictions including limits on how much medication should be administered in a given time period. Some of the limits are based on a daily, weekly, 21 day period, 30 day period, annual or lifetime basis, for example. The effect on a patient of exceeding these limits may range from mild to life threatening. These limits may be reached over multiple patient encounters (e.g., comprising visits with one or more healthcare providers or pharmacies and involving multiple different prescriptions for the same or different medications, including refills).

Known systems track daily maximum doses and issue associated alerts to pharmacy systems. A pharmacist is responsible for contacting an appropriate individual to verify and solicit changes required to prevent individual doses from exceeding a patient cumulative maximum. Notification of an individual physician is typically manual such as by adding printed information to a patient chart. Other known systems, as components of Computerized Physician Order Entry (CPOE) systems use simple limit rules or drug interaction checking to notify an ordering physician if individual doses approach a patient cumulative maximum. While the known system functions are valuable, they do not accommodate multiple encounters and different methods of notification including comprehensive User configurable notification functions for multiple recipients The known systems also do not provide worklist integration with a Healthcare Information System (HIS), medication administration and ordering systems and pharmacy systems. Known systems do not keep dosage information over multiple different patient encounters with one or more healthcare provider systems so data may be limited to dosage delivered during a current visit or encounter. Further, known systems depend on manual notification and manual limit monitoring and use manual methods subject to errors, forgotten notifications, missed limit checking and missed patient checking. Such manual systems are vulnerable to error and omission of critical clinical information from multiple sources and locations. A system according to invention principles addresses these deficiencies and related problems.

### SUMMARY OF THE INVENTION

The inventor has advantageously recognized it is desirable to provide a system for automatically tracking patient medication dosages over multiple encounters with one or more healthcare providers and automatically alerting pertinent clinicians through different methods. A system integrates drug dosage administration functions, clinical information records and a workflow system providing clinical notifications (e.g., via Short Messaging Service (SMS), Multimedia Messaging Service (MMS), e-mail, fax, instant message or via a voice response unit) and personnel and device worklists. The system monitors patient cumulative medication intake using at least one repository of information identifying medications and associated cumulative dosage limits for different time periods and for different types of patient. A monitoring processor, in response to a detected administration event indicating an additional dosage of a particular medication is to be administered to a particular patient, automatically determines whether a cumulative dosage, including the additional dosage, of the particular medication of the particular patient exceeds a cumulative dosage limit over a time period using information derived from the at least one repository. A notification processor automatically initiates generation of an alert message to at least one healthcare worker using one or more selected communication methods. The selected healthcare worker and the selected communication method(s) are identified using predetermined notification configuration information.

### BRIEF DESCRIPTION OF THE DRAWING

FIG. 1 shows a system for monitoring patient cumulative medication intake, according to invention principles.

FIG. 2 shows a notification system used by the system for monitoring patient cumulative medication intake, according to invention principles.

FIG. 3 shows a user interface display image presenting a history of medications, running cumulative dosages, dosage threshold limits and associated notification alert messages for a particular patient, according to invention principles.

FIG. 4 shows a user interface display image presenting warning text with details of notifications for one or more clinicians and a link to an item on a clinician worklist, according to invention principles.

FIG. 5 shows a flowchart of a process performed by a system for monitoring patient cumulative medication intake, according to invention principles.

### DETAILED DESCRIPTION OF THE INVENTION

A system integrates drug dosage administration functions, clinical information records and a workflow system. The workflow system manages personnel and device tasks by automatically adding tasks to, or removing tasks from, personnel or device task worklists as well as editing the tasks on the worklists. The workflow system further provides clinical, e.g., alert notifications (such as via SMS, MMS, e-mail, fax, instant message or via a voice response unit) to devices and personnel. The system keeps track of historical medication administrations and cumulative medication doses over various periods of time up to an entire patient lifetime. In response to a cumulative medication maximum limit threshold being reached as indicated by an associated executed rule, a combination of clinicians, administrators or other individuals are automatically notified via a worklist, for example. The system is used to help prevent accidentally exceeding medication dosage limits and to improve patient safety and quality of care.

The system automatically tracks cumulative medication dosages of patients and associated maximum cumulative limits and provides links to one or more worker or device worklists and user configurable notification communication methods. A task representative item may be automatically communicated to a clinician by e-mail, fax, instant message, cell phone, SMS, MMS or by placing the item on the clinician worklist. Alert notification messages are sent to a user determined list of clinicians for each medication. The clinicians may include one or more of the following, attending physician, nurses, nurse practitioners, referring physician, consulting physician, family physician, pharmacist, medical site quality control officer, facility risk management personnel or another named individual. The system integrates medication dose administration activities with a clinician centric worklist and other alerting options by using user determined cumulative medication maximum dosage limits to trigger notifications. The system allows individual clinicians to identify how they would like to be notified, how many ways they would like to be notified and at what user-defined intervals they would like to receive the notification. The system also allows a user to suspend or discontinue notifications. The system further facilitates prevention of cumulative medication maximum dosage limits being accidentally exceeded by automatically notifying appropriate clinicians when a drug intake is close to reaching or exceeding predetermined limits. A notification message is generated that indicates specific cumulative medication (e.g., drug) dosage intake approaching or exceeding a maximum limit per medication or ingredient, in response to predetermined notification configuration threshold limit information as well as predetermined configuration information identifying one or more clinicians to be notified and associated notification communication methods.

A processor, as used herein, operates under the control of an executable application to (a) receive information from an input information device, (b) process the information by manipulating, analyzing, modifying, converting and/or transmitting the information, and/or (c) route the information to an output information device. A processor may use, or comprise the capabilities of, a controller or microprocessor, for example. The processor may operate with a display processor or generator. A display processor or generator is a known element for generating signals representing display images or portions thereof. A processor and a display processor may comprise a combination of hardware, firmware, and/or software.

An executable application, as used herein, comprises code or machine readable instructions for conditioning a processor to implement predetermined functions, such as those of an operating system a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A user interface (UI), as used herein, comprises one or more display images, generated by a display processor and enabling user interaction with a processor or other device and associated data acquisition and processing functions. The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen, stylus or any other means allowing a user to provide data to a processor. The processor, under control of an executable procedure or executable application manipulates the UI display images in response to the signals received from the input devices In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity. Workflow comprises a sequence of tasks performed by a device or worker or both. An object or data object comprises a grouping of data, executable instructions or a combination of both or an executable procedure. A document or record comprises a compilation of data in electronic or paper form.

A workflow processor, as used herein, processes data to determine tasks to add to a task list, remove from a task list or modifies tasks incorporated on, or for incorporation on, a task list. A task list is a list of tasks for performance by a worker or device or a combination of both. A workflow processor may or may not employ a workflow engine. A workflow engine, as used herein, is a processor executing in response to predetermined process definitions that implement processes responsive to events and event associated data. The workflow engine implements processes in sequence and/or concurrently, responsive to event associated data to determine tasks for performance by a device and or worker and for updating task lists of a device and a worker to include determined tasks. A process definition is definable by a user and comprises a sequence of process steps including one or more, of start, wait, decision and task allocation steps for performance by a device and or worker, for example. An event is an occurrence affecting operation of a process implemented using a process definition.

A Workflow Management System is a software system that manages processes. It includes a process definition function that allows users to define a process that should he followed, an Event Monitor, which captures events from a Healthcare Information System and communicates the results to the Workflow Management System. A processor in the Management System tracks which processes are running, for which patients, and what step needs to be executed next, according to a process definition. The Management System includes a procedure for notifying clinicians of a task to be performed, through their worklists and a procedure for allocating and assigning tasks to specific users or specific teams. A document or record comprises a compilation of data in electronic form and is the equivalent of a paper document and may comprise a single, self-contained unit of information.

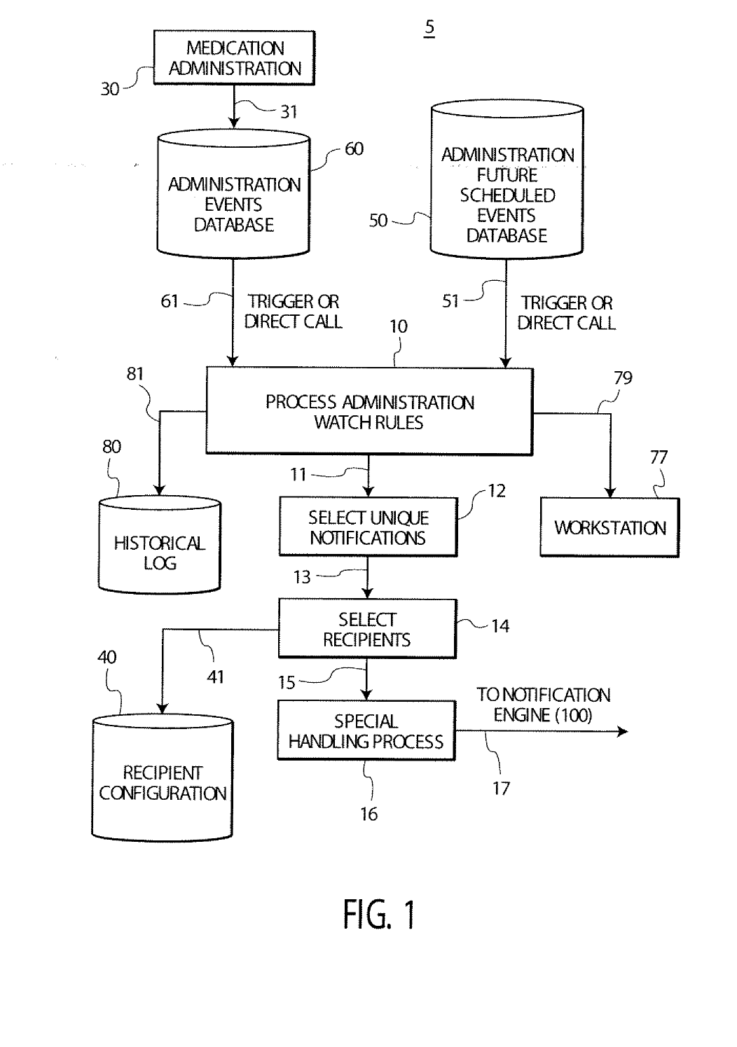

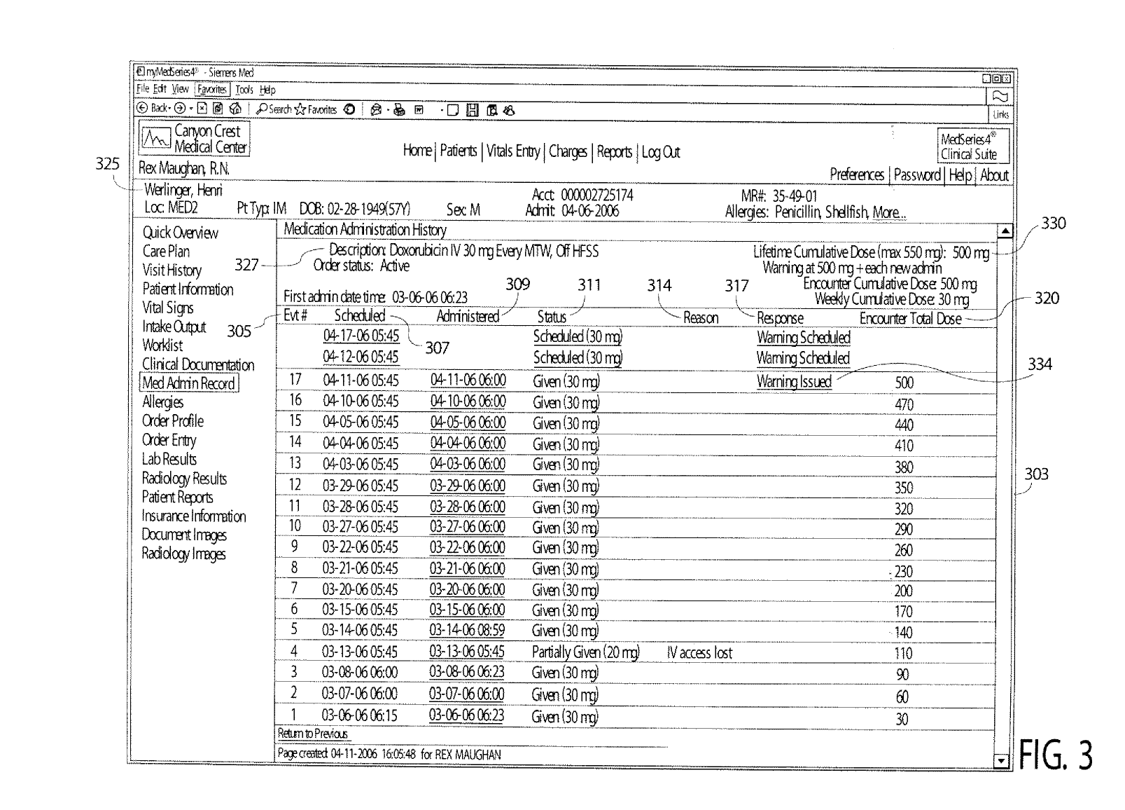

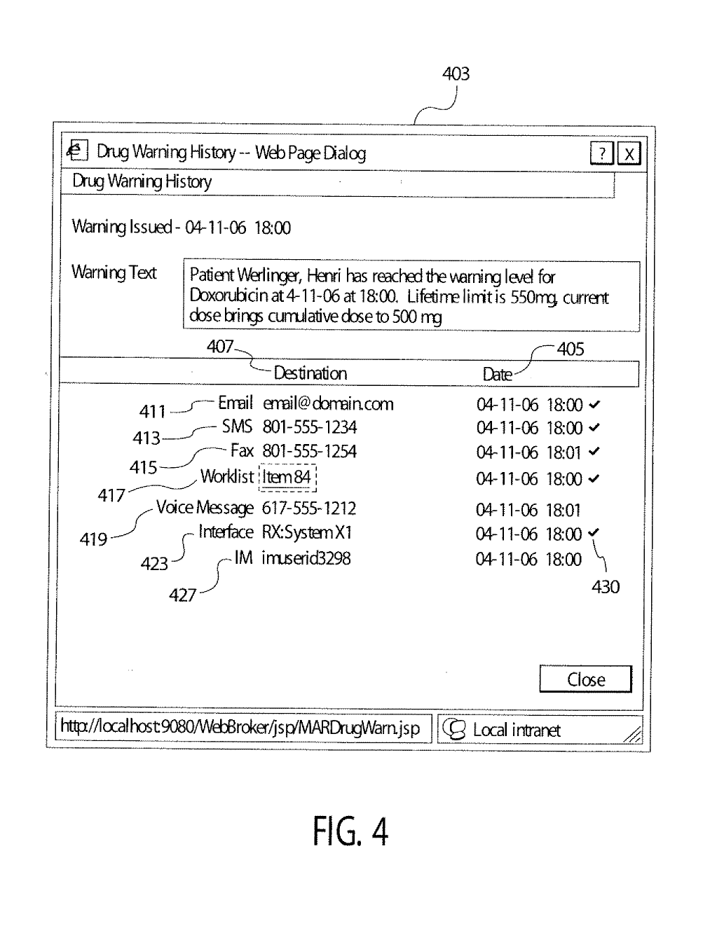

FIG. 1 shows system 5 for monitoring patient cumulative medication intake System 5 includes at least one repository of information (databases 40, 50 and 60) identifying medications and associated cumulative dosage limits for different time periods and for different types of patient. The different types of patient include patient based on age (e.g., infants have different dose limits than adults), weight, gender, medical condition. A monitoring processor (processor 10) processes administration watch rules. Processor 10, in response to a detected administration event indicating an additional dosage of a particular medication is to be administered to a particular patient, automatically determines whether a cumulative dosage, including the additional dosage, of the particular medication of the particular patient exceeds a cumulative dosage limit over a time period using information derived from the at least one repository. Processor 10 communicates via interface 79 with workstation 77 including a display generator for generating data representing display images for presentation on workstation 77. The display images include the images of FIGS. 3 and 4 indicating cumulative total medication dose, for example. A notification processor (unit 1 of FIG. 1) automatically initiates generation of an alert message to at least one selected healthcare worker using a selected communication method The selected healthcare worker and the selected communication method are identified using predetermined notification configuration information.

Data identifying an administration event is automatically stored in Administration Events Database 60 in response to data entry by a clinician. The administration event may comprise, administering a medication, holding a medication administration, revising a medication administration or receiving data identifying a medication administration from an external system interface though medication administration process 30. The details of individual medication administrations are stored in the Administration Events Database 60 through interface 31. Database 60 holds data identifying medication administrations for medications and associated ingredients for the lifetime of a patient. Detailed information stored in database 60 includes data indicating date and time, medication, ingredient, date of administration, identifier of worker administering the medication, dose quantity and units, missed administrations, additional reason descriptions and flags for early, late or partially given medications.

In response to occurrence of an administration event and storage of data identifying an administration event in database 60, either a direct call 61 is communicated to administration watch rules processor 10 or a database trigger 61 is communicated which initiates execution of administration watch rules by processor 10. In response to a medication administration scheduler (not shown) storing data indicating future scheduled medication administration events in Administration Future Scheduled Events Database 50, either a direct call 51 is communicated to administration watch rules processor 10 or a database trigger 51 is communicated which initiates execution of administration watch rules by processor 10. Upon initiation of execution of the Administration Watch Rules, processor 10 checks, parses and validates individual rules prior to their execution. The Administration Watch Rules are initiated in response to particular event times including, for example, one or more of, at dose scheduling time where the next scheduled dose equals or exceeds predefined limits, after dose administration where the dose equals or exceeds predefined limits, at dose scheduling time where the total of the next user defined doses equals or exceeds predefined limits, at initial order time where a first administration equals or exceeds predefined limits and at dose scheduling time where dose(s) administered over a next predetermined number of hours (user defined hours) equals or exceeds predefined limits.

If at least one of the executed Administration Watch Rules provides a potential alert notification to be processed for communication to a device or worker, a list of potential notifications is passed to Select Unique Notifications processor 12 via interface 11. Select Unique Notifications processor 12 reviews potential notifications and eliminates replicate notifications. This prevents a user from receiving multiple notifications for the same detected dosage condition. Once redundant replicated potential notifications are eliminated, the updated potential notifications are passed to a Select Recipients process 14 through link 13. Select Recipients unit 14 processes received potential cumulative dosage alert notifications to determine the number of recipients that are to receive a notification using predetermined Recipient Configuration data from repository 40 acquired via link 41. If the configuration data indicates a notification is a critical notification, additional recipients may be selected such as recipients associated with risk management, quality control or administration for possible additional corrective action. Selected (non-replicated) and consolidated alert notifications are passed to Special Handling Processor 16 through interface 15. Special Handling Processor 16 examines individual alert notifications and removes any notifications that are indicated as being on hold or disabled. A finalized list of notifications is compiled by Processor 16 and passed through interface 17 to Notification Engine (processor) 100 for further processing. The activities of processor 16 are recorded in historical log 80 through link 81.

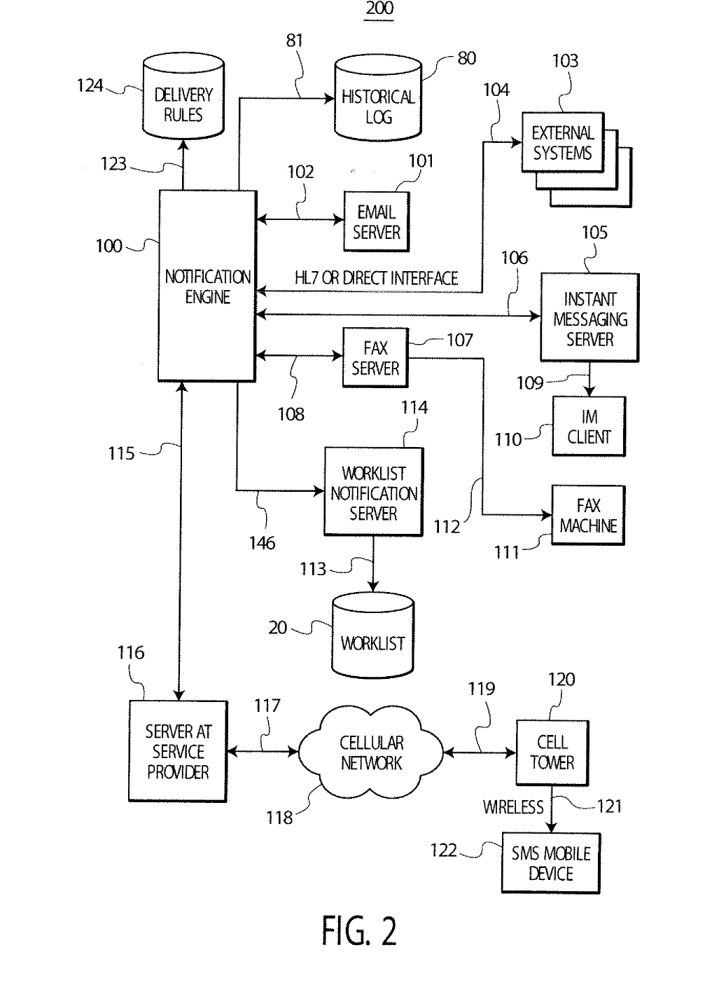

FIG. 2 shows notification system 200 used by the system for monitoring patient cumulative medication intake. Notification processor 100 receives the finalized list of notifications from processor 16 (FIG. 1) and processes individual notifications using delivery rules stored in Delivery Rules database 124 using link 123. Notification processor 100 processes an individual notification using the rules by comparing notification parameters indicating notification type, source and destination, for example, with stored data. The delivery rules may initiate generation of one or more notification messages for communication by one or more different communication methods. If Notification processor 100 determines that an individual notification needs to be communicated by email, the message contents and destination are generated by processor 100 and communicated to email server 101 through interface 102 for email communication e.g., via the Internet (as known). If Notification processor 100 determines that a notification message needs to be sent to an external system, processor 100 generates a notification message for communication to a particular destination and initiates communication of the message either through an. HL7 (HealthLevel7) based interface or direct interface through path 104 to one of multiple different external systems 103.

If Notification processor 100 determines that a notification is to be sent by Instant Messaging, processor 100 generates a notification message for communication to Instant Messaging Server 105 through interface 106. Instant Messaging Server 105 formats the message for an Instant Messaging (IN) Client 110 and sends it through interface 109 to IM client 110 via known IM protocol. If Notification processor 100 determines that a notification needs to be communicated by fax, processor 100 generates a notification message for communication to fax server 107 through interface 108. A fax is formatted and sent to fax machine 111 using interface or phone line 112 via fax communication (as known). If Notification processor 100 determines that a notification needs to be sent by Short Messaging Service (SMS) or as it is commonly known “cell phone text messaging” or by Multimedia Messaging Service (MMS), processor 100 generates a notification message for communication to service provider 116 through interface 115. Service provider 116 sends the message through the cellular network 118 via interface 117. The cellular network 118 finds a proper cellular communication tower 120 and provides the message through interface 119. The message is communicated to SMS or MMS Mobile device 122 through wireless connection 121. If Notification processor 100 determines that a notification needs to be included in one or more clinician worklists, processor 100 generates a notification message for communication to worklist notification server 114 through interface 146. Worklist Notification Server 114 incorporates entries on individual stored clinician worklists such as Worklist 20 through interface 113. The activities of Notification processor 100 are recorded in the historical log 80 through link 81.

Systems 5 and 200 provide specific notifications for medication administration and may be used as part of a medication administration record (MAR) system, a physician ordering system or as additional functionality for a worklist management system and medication administration information system. FIG. 3 shows user interface display image 303 presented on workstation 77 (FIG. 1) using data provided by processor 10. A user initiates display of image 303 by selecting to display an administration history from a Medications Administration Record web page. Display image 303 illustrates lifetime cumulative dose, encounter cumulative doses and weekly cumulative dose totals. For specific drugs or ingredients, the accumulation time frame may be set for biweekly, 21 day totals, 28 day totals or any other time frame described in hour intervals. Display image 303 presents a history of medication administrations, running cumulative dosages, dosage threshold limits and associated notification alert messages for a particular patient. Specifically, image 303 in column 307 indicates scheduled medication administrations of medication 327 in sequential order for patient 325 and actual administration time and date in column 309. A corresponding medication administration event number is identified in column 305. Further, column 311 indicates status of medication administration including whether administered, being administered, scheduled to be administered or partially given, for example and a corresponding reason for the status in column 314 (e.g., IV access lost). A total cumulative dosage of the administered medication is shown in column 320 and column 317 indicates associated issued notifications (e.g., event 17 on 4-11-06 @5.45) and notifications (alerts or warnings) that are scheduled to be issued in response to future medication administrations) as well as warnings scheduled based on future scheduled administration events. The warning indications use hyperlinks that are selectable to view details about issued or future scheduled warnings. Columns 320 and 317 indicate that a warning was issued when the patient cumulative dosage reached 500 mg within ten percent of the lifetime permitted total cumulative dose 330 (550 mg).

FIG. 4 shows user interface display image 403 presenting a history of warning notifications for a patient with details of various notifications made via different communication methods to one or more different clinicians and including a link (e.g., hyperlink) to an item on a clinician worklist. Display image 403 is presented in response to user selection of an issued warning hyperlink 334 in image 303 (FIG. 3). Display image 403 is presented on workstation 77 (FIG. 1) using data provided by processor 10. Specifically, display image 403 in column 405 indicates time and date of communication of alert notification messages of the cumulative dosage of FIG. 3 reaching 500 mg. Column 407 (FIG. 4) indicates the communication message type of corresponding messages identified in column 405. The communicated messages include email 411, SMS 413, Fax 415, voice 419, interface 423, IM 427 and an item communicated for inclusion in a worklist 417. The check marks, e.g., 430 indicate that successful notification has been completed. Communicated user notification message information of display image 403 is recorded in a historical log (e.g., log 80 FIG. 2).

Notification messages are communicated by system 2 by one or more of, e-mail (as a link to a secured system involving authentication and without communicating patient identification information), secured e-mail, fax, instant messaging, wireless Short Messaging Service (SMS) and Multimedia Messaging Service (MMS). Other methods used by system 200 communicate an interface transaction to a defined system, send an outgoing voice message (e.g., using calling software with an outgoing voice response unit (VRU), for example) and enable addition of an item to a clinician worklist. If a cumulative dosage value triggers more than one user alert notification rule such that communication of different alert notification messages may be initiated, processor 10 ensures only a single notification message is sent. This prevents a cumulative medication dose value meeting multiple alert criteria and generating superfluous multiple notification messages. Processor 10 enables a user to disable communication of notification messages for a particular clinician, patient, clinician and patient, medication, medication and clinician, health care location or facility, patient and medication and medication therapeutic class. A record of disabled notifications is stored in historical log 80 together with a user selectable reason identifying a user, date and time of notification received, location initiating disablement (usually IP address) with a link to the notification details.

If an alert notification is indicated as being critical, an additional notification message may be sent to additional individuals such as hospital administration personnel to limit patient risk through additional reviews and notifications. Processor 10 also enables a clinician to disable future drug notifications for a patient after reviewing delivered notification messages. Further, processor 10 enables an individual clinician to suspend notifications, for a period of time (hours, minutes, days, or months) for a specific patient and/or medication. In addition to the notifications, processor 10 initiates generation of reports for quality control or system improvement by a hospital facility, for example. The reports generated include, a report indicating alert notification messages of a clinician sorted by date of notification, a report indicating notification messages generated for a selected patient, report statistics showing the number of notification messages communicated using each notification method (i.e., how many e-mails, how many test messages, how many voice messages, for example). Another report indicates suspended notifications by day, or by patient or by clinician, for example.

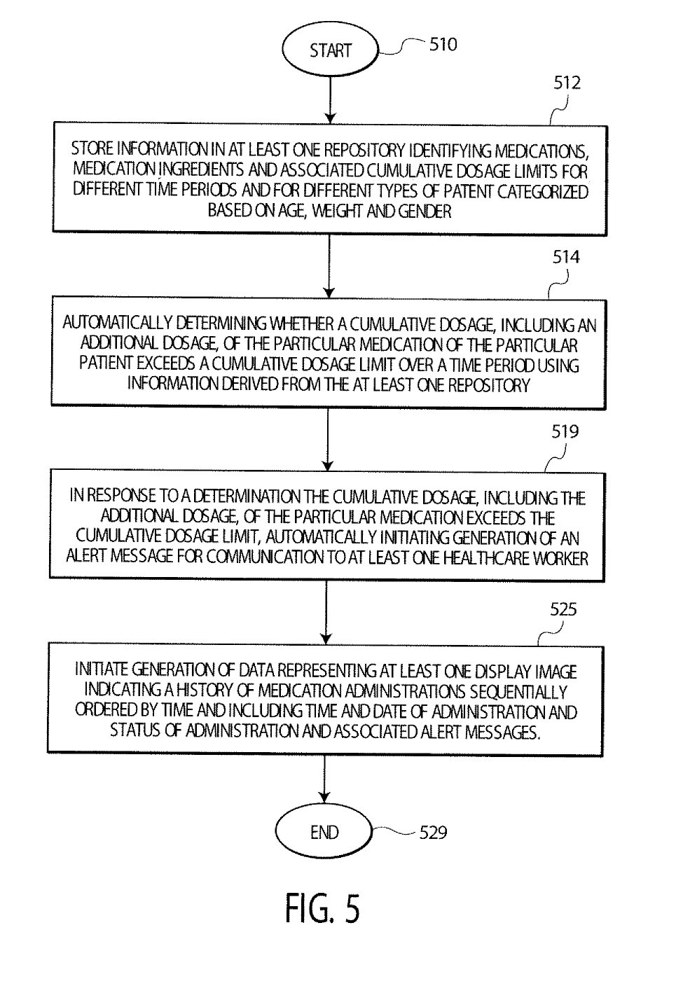

FIG. 5 shows a flowchart of a process performed by a system for monitoring patient cumulative medication intake. The steps of FIG. 5 may be performed automatically. In step 512 following the start at step 510, information is stored in at least one repository (e.g., repositories, 40, 50 FIG. 1). The information identifies medications and their ingredients and associated cumulative medication and ingredient dosage limits for different time periods and for different types of patient categorized based on age, weight and gender. The different time periods comprise multiple different time periods including at least two of, (a) daily (b) weekly, (c) a 21 day period (d) a 30 day period, (e) annually and (f) over a patient lifetime. The different types of patient comprise patients categorized based on at least one of, age, weight and gender. In step 514 in response to a detected administration event indicating an additional dosage of a particular medication or ingredient is to be administered to a particular patient, monitoring processor 10 (FIG. 1) automatically determines whether a cumulative dosage, including the additional dosage, of the particular medication or ingredient of the particular patient exceeds a cumulative dosage limit over a time period using information derived from the at least one repository. The detected administration event comprises at least one of, detection of placing of an order for administration of the additional dosage of the particular medication (or ingredient) to the particular patient and detection of scheduled administration of the additional dosage of the particular medication (or ingredient) to the particular patient. Monitoring processor 10 further determines whether the cumulative dosage of different medications of different patients, including the particular medication of the particular patient, exceeds a cumulative dosage limit over the multiple different time periods.

In response to a determination the cumulative dosage, including the additional dosage of the particular medication or ingredient, exceeds the cumulative dosage limit, notification processor 100 (FIG. 2) in step 519, automatically initiates generation of an alert message for communication to at least one healthcare worker. Notification processor 100 automatically initiates generation of an alert message for communication to at least one selected healthcare worker using a selected communication method. Notification processor 100 automatically identifies and eliminates replicated alerts and initiates generation of a single consolidated alert message for communication to at least one selected healthcare worker. The selected healthcare worker and the selected communication method are identified using predetermined notification configuration information. In step 525 a display processor (in processor 10 or workstation 77 FIG. 1) initiates generation of data representing at least one display image indicating a history of medication administrations sequentially ordered by time and including time and date of administration and status of administration and associated alert messages. The status of administration indicates whether a medication was administered in full, partially or not administered. A display image includes a hyperlink associated with an alert message and in response to user selection of the hyperlink additional information concerning the alert message is displayed. A further display image also indicates alert messages of a particular clinician and different types of communication methods used for communicating the alert messages of the particular clinician. Other display images enable a user to disable alert notification for one or more of, a particular medication, a particular clinician, a particular facility and a particular time period and enable a user to configure, (a) recipients of alert notification messages, (b) methods of communication of alert notification messages and (c) a time period over which to compute a cumulative dosage. The process of FIG. 5 terminates at step 529.

The system and processes of FIGS. 1-5 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. Systems 5 and 200 for monitoring patient cumulative medication intake are applicable to monitoring other cumulative parameters with limits triggering action by workers or devices. The processes and applications may in alternative embodiments, be located on one or more (e.g., distributed) processing devices accessing a network linking the elements of FIGS. 1 and 2. Further, any of the functions and steps provided in FIGS. 1-5 may be implemented in hardware, software or a combination of both and may reside on one or more processing devices located at any location of a network linking the elements of FIGS. 1 and 2 or another linked network including the Internet.

What is claimed is:

1. A system for monitoring patient cumulative medication intake, comprising:
at least one repository of information identifying medications and associated cumulative dosage limits for different time periods and for different types of patient;
a monitoring processor for, in response to a detected administration event indicating an additional dosage of a particular medication is to be administered to a particular patient, automatically determining whether a cumulative dosage, including said additional dosage, of said particular medication of said particular patient exceeds a cumulative dosage limit over a time period using information derived from said at least one repository; and
a notification processor for, in response to a determination said cumulative dosage, including said additional dosage, of said particular medication exceeds said cumulative dosage limit, automatically initiating generation of an alert message for communication to at least one selected healthcare worker using a selected communication method, said selected healthcare worker and said selected communication method being identified using predetermined notification configuration information.

2. A system according to claim 1, wherein
said different time periods comprise a plurality of different time periods including at least two of (a) daily, (b) weekly, (c) a 21 day period, (d) a 28 day period, (e) a 30 day period, (f) annually and (g) over a patient lifetime and
said monitoring processor determines whether said cumulative dosage of said particular medication of said particular patient exceeds a cumulative dosage limit over said plurality of different time periods.

3. A system according to claim 1, wherein
said different time periods comprise a plurality of different time periods including at least two of, (a) daily, (b) weekly, (c) a 21 day period, (d) a 28 day period (e) a 30 day period, (f) annually and (g) over a patient lifetime and
said monitoring processor determines whether a cumulative dosage of different medications of different patients exceeds a cumulative dosage limit over said plurality of different time periods.

4. A system according to claim 1, wherein
said different types of patient comprise patients categorized based on at least one of, (a) age, (b) weight and (c) gender.

5. A system according to claim 1, wherein
said notification processor identifies and eliminates replicated alerts and initiates generation of a single consolidated alert message for communication to said at least one selected healthcare worker.

6. A system according to claim 1, wherein
said information identifies ingredients of medications and associated cumulative dosage limits of ingredients for different time periods and for different types of patient and
said monitoring processor automatically determines whether a cumulative dosage, including said additional dosage of an ingredient of a particular medication of said particular patient exceeds a cumulative dosage limit over a time period using information derived from said at least one repository.

7. A system according to claim 1, including
a display processor for initiating generation of data representing at least one display image indicating a history of medication administrations and associated alert messages.

8. A system according to claim 7, wherein
said at least one display image includes a hyperlink associate d with an alert message and in response to user selection of said hyperlink additional information concerning said alert message is displayed.

9. A system according to claim 7, wherein
said at least one display image includes a history of medication administrations sequentially ordered by time and including time and date of administration and status of administration.

10. A system according to claim 9, wherein
said status of administration indicates whether a medication was administered in full, partially or not administered.

11. A system according to claim 1, including
a display processor for initiating generation of data representing at least one display image indicating alert messages of a particular clinician and different types of communication methods used for communicating said alert messages of said particular clinician.

12. A system according to claim 1, wherein
said detected administration event comprises at least one of, (a) detection of placing of an order for administration of said additional dosage of said particular medication to said particular patient and (b) detection of scheduled administration of said additional dosage of said particular medication to said particular patient.

13. A system according to claim 1, including
a display processor for initiating generation of data representing at least one display image enabling a user to disable alert notification for at least one of, (a) a particular medication, (b) a particular clinician, (c) a particular facility and (d) a particular time period.

14. A system according to claim 1, including
a display processor for initiating generation of data representing at least one display image enabling a user to configure (a) recipients of alert notification messages, (b) methods of communication of alert notification messages and (c) a time period over which to compute a cumulative dosage.

15. A system for monitoring patient cumulative medication intake, comprising:
at least one repository of information identifying ingredients of medications and associated ingredient cumulative dosage limits for different time periods and for different types of patient;
a monitoring processor for, in response to a detected administration event indicating an additional dosage of a particular ingredient has been administered to a particular patient, automatically determining whether a cumulative dosage, including said additional dosage, of said particular ingredient administered to said particular patient exceeds a cumulative dosage limit over a time period using information derived from said at least one repository; and
a notification processor for automatically initiating generation of an alert message to at least one healthcare worker using a selected communication method, said selected healthcare worker and said selected communication method being identified using predetermined notification configuration information.

16. A system for monitoring patient cumulative medication intake, comprising:
at least one repository of information identifying medications and associated cumulative dosage limits for different time periods and for different types of patient categorized based on age, weight and gender;
a monitoring processor for, in response to a detected administration event indicating an additional dosage of a particular medication is to be administered to a particular patient, automatically determining whether a cumulative dosage, including said additional dosage, of said particular medication of said particular patient exceeds a cumulative dosage limit over a time period using information derived from said at least one repository;
a notification processor for, in response to a determination said cumulative dosage, including said additional dosage, of said particular medication exceeds said cumulative dosage limit, automatically initiating generation of an alert message for communication to at least one healthcare worker; and
a display processor for initiating generation of data representing at least one display image indicating a history of medication administrations sequentially ordered by time and including time and date of administration and status of administration and associated alert messages.

17. A system according to claim 16, wherein
said notification processor automatically initiates generation of an alert message for communication to at least one selected healthcare worker using a selected communication method, said selected healthcare worker and said selected communication method being identified using predetermined notification configuration information.
